(19) United States
(12) Patent Application Publication
(10) Pub. No.: US 2026/0007857 A1
(43) Pub. Date: Jan. 8, 2026

(54) GUIDE CATHETER WITH FLAT SEAL FOR PREVENTING AIR INGRESS

(71) Applicant: BOSTON SCIENTIFIC SCIMED, INC., MAPLE GROVE, MN (US)

(72) Inventors: Aman Kumar Verma, Lakhisarai (IN); Ryan Robert Davis, Plymouth, MN (US); Peeyush Tomar, Muzaffarnagar (IN); Neeraj Kumar Sharma, New Delhi (IN); Manoj Kumar, New Delhi (IN); Aniket Kajal, Kurukshetra (IN); Shubham Gupta, Gurgaon (IN)

(73) Assignee: BOSTON SCIENTIFIC SCIMED, INC., MAPLE GROVE, MN (US)

(21) Appl. No.: 19/257,934

(22) Filed: Jul. 2, 2025

Related U.S. Application Data
(60) Provisional application No. 63/666,954, filed on Jul. 2, 2024.

(51) Int. Cl. A61M 25/00

(52) U.S. Cl. CPC A61M 25/0097; A61M 25/005

(57) ABSTRACT

A guide catheter that is adapted to accommodate a left atrial appendage closure (LAAC) device being advanced towards a left atrial appendage (LAA) includes an elongate shaft extending from a proximal region to a distal region and a guide catheter hub that is coupled to the proximal region of the elongate shaft. The guide catheter hub includes a hub body, a Tuohy seal disposed relative to the hub body, a flat seal disposed relative to the hub body, and a mechanism disposed relative to the hub body that is adapted to releasably open the flat seal.

### CROSS REFERENCE TO RELATED APPLICATIONS

This application claims the benefit of priority of U.S. Provisional Application No. 63/666,954 filed Jul. 2, 2024, the entire disclosure of which is hereby incorporated by reference.

### TECHNICAL FIELD

The disclosure relates generally to medical devices and more particularly to guide catheters including guide catheter hubs that provide a fluid-tight access through the guide catheter hub for other medical devices.

### BACKGROUND

A wide variety of intracorporeal medical devices have been developed for medical use, for example, intravascular use. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in a guide catheter for accommodating a left atrial appendage closure (LAAC) device being advanced towards a left atrial appendage (LAA). The guide catheter includes an elongate shaft extending from a proximal region to a distal region and a guide catheter hub that is coupled to the proximal region of the elongate shaft. The guide catheter hub includes a hub body, a Tuohy seal disposed relative to the hub body, a flat seal disposed relative to the hub body, and a mechanism disposed relative to the hub body and adapted to releasably open the flat seal.

Alternatively or additionally, the flat seal may be disposed within the hub body.

Alternatively or additionally, the mechanism may be disposed within the hub body.

Alternatively or additionally, the mechanism includes an internal actuation mechanism that is disposed within the hub body and an external actuation mechanism that is disposed outside of the hub body and that is adapted to engage with and move the internal actuation mechanism.

Alternatively or additionally, the internal actuation mechanism may be biased to a position in which the flat seal is closed.

Alternatively or additionally, the guide catheter may further include a biasing member that is adapted to bias the internal actuation mechanism to the position in which the flat seal is closed.

Alternatively or additionally, the guide catheter may further include a proximal nut that is adapted to reversibly close the Tuohy seal.

Alternatively or additionally, the guide catheter may further include an actuation hub that is disposed proximal of the hub body.

Alternatively or additionally, the Tuohy seal and the flat seal may both be disposed within the actuation hub.

Alternatively or additionally, the mechanism includes a threaded distal pusher, a threaded proximal pusher, and a nut having a first threaded surface that is adapted to engage the threaded distal pusher and a second threaded surface that is adapted to engage the threaded proximal push. The nut includes a proximally facing extension. Moving the nut in a first direction may cause the threaded distal pusher to engage and close the Tuohy seal and moving the nut in a second direction may cause the proximally facing extension to engage and open the flat seal.

Alternatively or additionally, the Touhy seal may be disposed within the hub body and the flat seal may be disposed within the actuation hub.

Alternatively or additionally, the actuation hub may include an inner hub including a tab extending radially outwardly from the inner hub and an outer hub accommodating the inner hub within the outer hub. The outer hub includes a slot adapted to allow the tab to slide within the slot.

Alternatively or additionally, the mechanism includes a nut having a threaded surface and a threaded member associated with the inner hub. Rotating the nut in a first direction may cause the threaded member to engage and close the Tuohy seal. Rotating the nut in an opposing section direction may cause the inner hub to translate relative to the outer hub as the tab slides within the slot, the inner hub engaging and opening the flat seal.

Alternatively or additionally, the mechanism may further include a spring that biases the inner hub to a position in which the inner hub does not engage the flat seal.

Another example may be found in a guide catheter for accommodating a left atrial appendage closure (LAAC) device being advanced towards a left atrial appendage (LAA). The guide catheter includes an elongate shaft extending from a proximal region to a distal region and a guide catheter hub that is coupled to the proximal region of the elongate shaft. The guide catheter hub includes a hub body, a first seal that is disposed within the hub body and is adapted to avoid air ingress, an internal actuation mechanism that is disposed within the hub body and is adapted to reversibly open the first seal, a second seal that is disposed within the hub body and is adapted to provide high pressure hemostasis, and an external actuation mechanism that is adapted to engage with and move the internal actuation mechanism.

Alternatively or additionally, the internal actuation mechanism may be biased to a position in which the first seal is closed.

Alternatively or additionally, the internal actuation mechanism may be further adapted to reversibly close the second seal.

Alternatively or additionally, the guide catheter may further include a proximal nut that is adapted to reversibly close the second seal.

Another example may be found in a guide catheter for accommodating a left atrial appendage closure (LAAC) device being advanced towards a left atrial appendage (LAA). The guide catheter includes an elongate shaft extending from a proximal region to a distal region and a guide catheter hub that is coupled to the proximal region of the elongate shaft. An actuation hub is disposed proximally of the guide catheter hub, a Tuohy seal is disposed relative to the hub body, a flat seal is disposed within the actuation hub. The guide catheter includes a mechanism that is adapted to selectively engage and close the Tuohy seal or open the flat seal.

Alternatively or additionally, actuating the mechanism in a first direction may cause the mechanism to engage and close the Tuohy seal, and actuating the mechanism in a second direction may cause the mechanism to engage and open the flat seal.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 provides an illustrative example of part of a procedure for implanting an LAAC (left atrial appendage closure) device, which includes a guide catheter used in combination with an elongate dilator;

FIG. 2 is a perspective view of an illustrative guide catheter;

FIG. 3 is a cross-sectional view of the illustrative guide catheter, taken along the line 3-3 of FIG. 2;

FIG. 4 is an exploded perspective view of a guide catheter hub forming part of the illustrative guide catheter of FIG. 2;

FIG. 5 is a perspective view of an illustrative flat seal used in the illustrative guide catheter of FIG. 2;

FIG. 6 is a perspective view of an illustrative guide catheter;

FIG. 7 is a cross-sectional view taken along the line 7-7 of FIG. 6;

FIG. 8 is an exploded perspective view of the illustrative guide catheter of FIG. 6;

FIG. 9 is a perspective view of an illustrative guide catheter;

FIG. 10 is a cross-sectional view taken along the line 10-10 of FIG. 9;

FIG. 11 is a perspective view of an illustrative flat seal used in the illustrative guide catheter of FIG. 9;

FIG. 12 is a perspective view of an illustrative guide catheter;

FIG. 13 is a cross-sectional view taken along the line 13-13 of FIG. 12; and

FIG. 14 is a perspective view of an illustrative flat seal used in the illustrative guide catheter of FIG. 12.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to seale, wherein like reference numerals indicate like elements throughout the several views. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

A variety of medical procedures may include the use of two different medical devices that are used in combination. As an example, a first medical device may be used in combination with a second medical device in order to gain access to a particular treatment site and then to carry out an appropriate treatment at the particular treatment site. In some cases, a guidewire may be advanced through the vasculature to reach a particular treatment site. A first medical device may be advanced over the guidewire, and then a second medical device may be advanced through the first medical device. In some instances, there may be a desire to be able to advance and control the first medical device and the second medical device as an assembly, meaning that both the first medical device and the second medical device may be moved, advanced or withdrawn axially without the user being required to separately hold both the first medical device and the second medical device at the same time. In some instances, the first medical device and the second medical device may be rotated as an assembly, meaning that the first medical device and the second medical device may be rotated together without the user being required to separately hold both the first medical device and the second medical device at the same time. In some instances, there can be advantages to being able to advance or rotate the first medical device and the second medical device together, particularly when the first medical device and the second medical device have curved distal regions and/or are steerable.

An illustrative but non-limiting example of a medical procedure that may utilize a first medical device and a second medical device in combination may include reaching the left atrium in order to implant a left atrial appendage closure (LAAC) device. In particular, a first medical device such as a guide catheter may be used in combination with a second medical device such as an elongate dilator. It will be appreciated that two such medical devices may be used in combination in performing any of a variety of different medical procedures. Gaining access to the left atrium in order to deliver and implant an LAAC is merely an example of using a first medical device in combination with a second medical device.

In some instances, a guide catheter may be adapted to accommodate a left atrial appendage closure (LAAC) device that is being advanced towards a left atrial appendage (LAA). The guide catheter includes an elongate shaft extending from a proximal region to a distal region and a guide catheter hub that is coupled to the proximal region of the elongate shaft. The guide catheter hub includes a hub body, a Tuohy seal disposed relative to the hub body, a flat seal disposed relative to the hub body, and a mechanism disposed relative to the hub body that is adapted to releasably open the flat seal.

In some cases, the flat seal may be disposed within the hub body. In some cases, the mechanism may be disposed within the hub body. As an example, the mechanism may include an internal actuation mechanism that is disposed within the hub body and an external actuation mechanism that is disposed outside of the hub body and that is adapted to engage with and move the internal actuation mechanism. In some cases, the internal actuation mechanism may be biased to a position in which the flat seal is closed. The guide catheter may further include a biasing member that is adapted to bias the internal actuation mechanism to the position in which the flat seal is closed. In some cases, the guide catheter may further include a proximal nut that is adapted to reversibly close the Tuohy seal.

In some cases, the guide catheter may further include an actuation hub that is disposed proximal of the hub body. In some cases, the Tuohy seal and the flat seal may both be disposed within the actuation hub. In some cases, the mechanism may include a threaded proximal pusher, a threaded distal pusher, and a nut having a first threaded surface that is adapted to engage the threaded proximal pusher and a second threaded surface that is adapted to engage the threaded distal push. The nut includes a proximally facing extension. Advancing the nut in a first direction may cause the threaded proximal pusher to engage and close the Tuohy seal and advancing the nut in a second direction may cause the proximally facing extension to engage and open the flat seal.

In some cases, the Touhy seal may be disposed within the hub body and the flat seal may be disposed within the actuation hub. The actuation hub may include an inner hub having a tab extending radially outwardly from the inner hub and an outer hub that accommodates the inner hub within the outer hub. The outer hub includes a slot that is adapted to allow the tab to slide within the slot. In some cases, the mechanism may include a nut having a threaded surface and a threaded member that is associated with the inner hub. Advancing the nut in a first direction may cause the threaded member to engage and close the Tuohy seal and advancing the nut in an opposing section direction may cause the inner hub to translate relative to the outer hub as the tab slides within the slot, the inner hub engaging and opening the flat seal. In some cases, the mechanism may further include a spring that biases the inner hub to a position in which the inner hub does not engage the flat seal.

In some instances, a guide catheter may be adapted for accommodating a left atrial appendage closure (LAAC) device being advanced towards a left atrial appendage (LAA). The guide catheter includes an elongate shaft extending from a proximal region to a distal region and a guide catheter hub that is coupled to the proximal region of the elongate shaft. The guide catheter hub includes a hub body, a first seal that is disposed within the hub body and that is adapted to avoid air ingress, an internal actuation mechanism that is disposed within the hub body and that is adapted to reversibly open the first seal, a second seal that is disposed within the hub body and that is adapted to provide high pressure hemostasis, and an external actuation mechanism that is adapted to engage with and move the internal actuation mechanism.

In some cases, the internal actuation mechanism may be biased to a position in which the first seal is closed. In some cases, the internal actuation mechanism may be further adapted to reversibly close the second seal. The guide catheter may further include a proximal nut that is adapted to reversibly close the second seal.

In some instances, a guide catheter may be adapted for accommodating a left atrial appendage closure (LAAC) device being advanced towards a left atrial appendage (LAA). The guide catheter includes an elongate shaft extending from a proximal region to a distal region, a guide catheter hub that is coupled to the proximal region of the elongate shaft, and an actuation hub that is disposed proximally of the guide catheter hub. A Tuohy seal is disposed relative to the hub body and a flat seal is disposed within the actuation hub. A mechanism is adapted to selectively engage and close the Tuohy seal or open the flat seal. In some cases, actuating the mechanism in a first direction may cause the mechanism to engage and close the Tuohy seal and actuating the mechanism in a second direction may cause the mechanism to engage and open the flat seal.

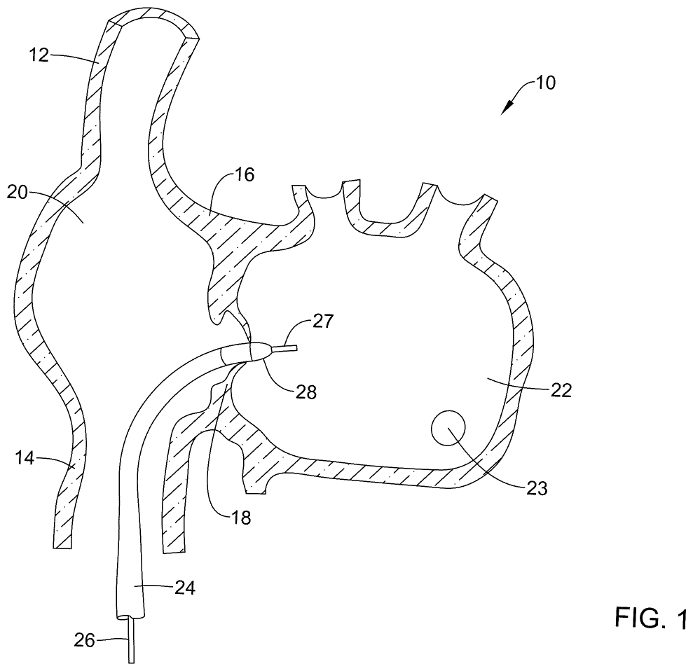

FIG. 1 provides a schematic view of a portion of a person's heart 10, including a superior vena cava 12, an inferior vena cava 14, a septum 16, an atrial septum 18, a right atrium 20 and a left atrium 22. In some cases, the left atrium 22 may include a left atrial appendage (LAA) 23. A composite medical device including a guide catheter 24 may be advanced over a guidewire 26. In some cases, the guidewire 26 may be an RF guidewire that is adapted to utilize RF (radio frequency) energy to cauterize, using a cautery tip 27. In some cases, for example, an RF guidewire may be used to form a small aperture in or near the atrial septum 18. In some cases, a guidewire may have a sharp distal end that may be used to form a puncture.

The puncture through the atrial septum 18 can be done from a position within the right atrium 20. By forming an aperture through the atrial septum 18, it is possible to reach the left atrium 22 from the relative safety of the right side of the heart. In some cases, an elongate medical device including an elongate dilator 28 may be advanced over the guidewire 26, and within the guide catheter 24. Once an aperture has been formed within the atrial septum 18, the elongate dilator 28 may be advanced over the guidewire and through the aperture in order to widen the aperture. From there, the elongate dilator 28 and guidewire 26 may be removed in order to allow a delivery device carrying an LAAC (left atrial appendage closure) device to be advanced through the guide catheter 24. A variety of devices may be advanced through the guide catheter 24 in order to reach the LAA 23. An illustrative example of a suitable LAAC device includes the Watchman FLX™ LAAC device commercially available from Boston Scientific Corporation.

In some cases, an assembly including the guide catheter 24 and the elongate dilator 28 may be advanced through the inferior vena cava 14 in order to reach the right atrium 20. It will be appreciated that this can represent a tortuous path through the vasculature. In some cases, the guide catheter 24 and/or the elongate dilator 28 may be adapted to have a curved distal end in order to facilitate steering. It will be appreciated that being able to hold the guide catheter 24 and the elongate dilator 28 from relative rotation therebetween may be beneficial in steering the devices through the anatomy. Additional details pertaining to providing a rotational lock between a first medical device and a second medical device may be found, for example, in U.S. patent application Ser. No. 18/439,047, filed Feb. 12, 2024 and entitled Multi-Part Medical Devices With Locking Mechanism, which application is incorporated by reference herein in its entirety.

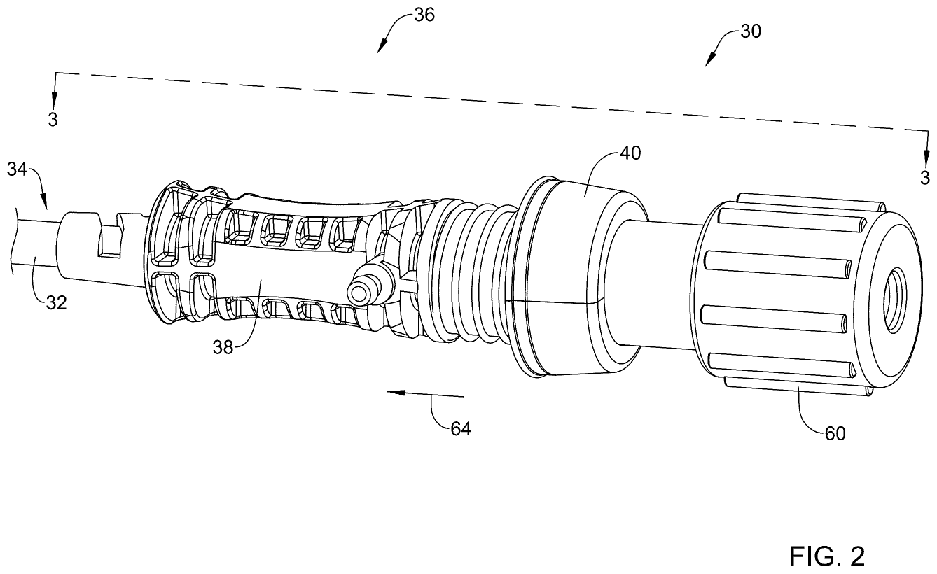

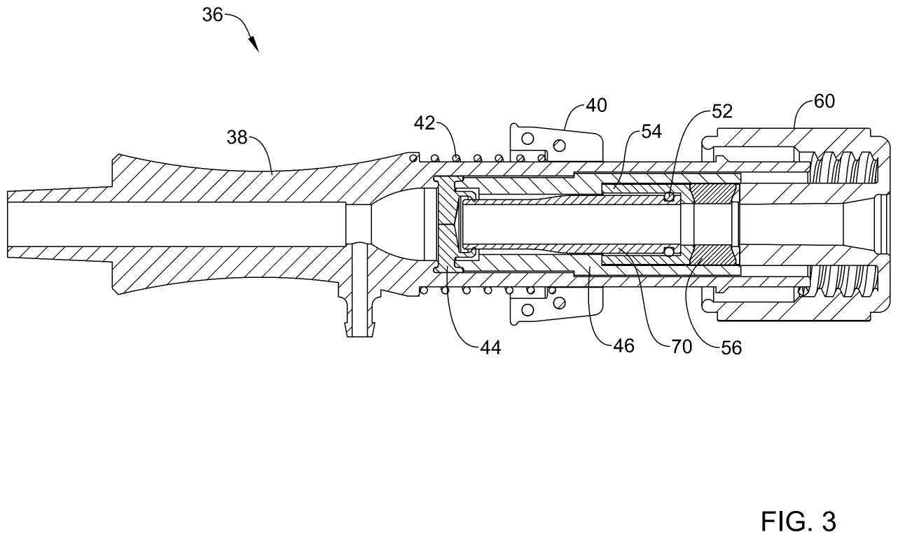

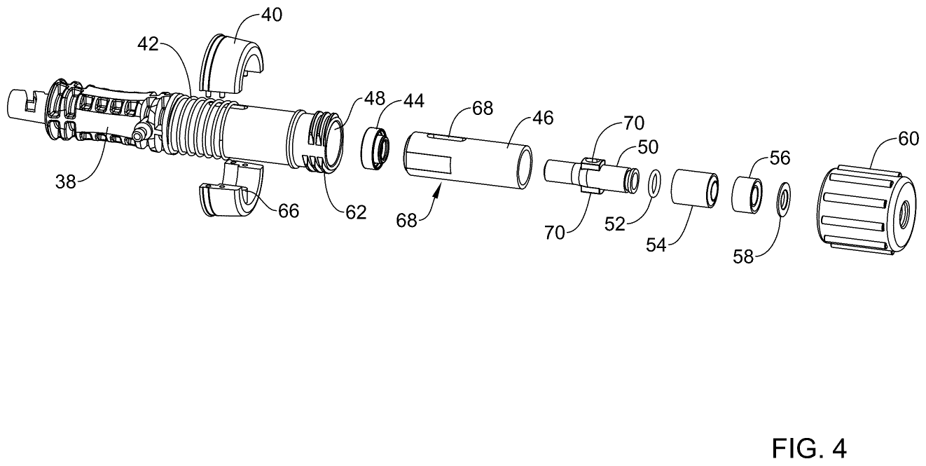

FIG. 1 shows a distal portion of the guide catheter 24. FIGS. 2 through 14 provide examples of guide catheters that may be used as the guide catheter 24 in accessing the LAA 23. In some cases, the guide catheters shown in FIGS. 2 through 14 provide benefits such as improved sealing and reduced possibility of air infiltration into the guide catheter 24. FIG. 2 is a perspective view of an illustrative guide catheter 30. The guide catheter 30 may be considered as being an example of the guide catheter 24 partially shown in FIG. 1. FIG. 3 is a cross-sectional view taken along the line 3-3 of FIG. 2. FIG. 4 is an exploded perspective view showing individual components within a guide catheter hub forming part of the guide catheter 30.

The guide catheter 30 includes an elongate shaft 32 that extends from a proximal region 34 to a distal region (as shown in FIG. 1). A guide catheter hub 36 is coupled to the proximal region 34 of the elongate shaft 32. The guide catheter hub 36 includes a hub body 38. A lever 40 is disposed about an exterior of the guide catheter hub 36. In some cases, the lever 40 may be considered as being an example of an external actuation mechanism. As shown, the lever 40 includes two halves that snap together or otherwise fit together. A spring 42 is disposed about the exterior of the guide catheter hub 36 and may be adapted to bias the lever 40 to a position that, as will be discussed, corresponds to a flat seal 44 being in a closed configuration. In some cases, the flat seal 44 may provide the guide catheter hub 36 with resistance to air ingress.

The guide catheter hub 36 includes a seal cavity 46 that itself is adapted to fit within an interior volume 48 within the hub body 38. A plunger 50 is adapted to fit within the seal cavity 46. In some cases, the plunger 50 is adapted to be moved into position to engage and thus open the flat seal 44. An O-ring 52 fits within an O-ring cavity 54. A Tuohy seal 56 and a washer 58 also fit into the interior volume 48. A proximal nut 60 is disposed at a proximal end of the guide catheter hub 36, and engages a threaded surface 62 that is formed on an exterior of the hub body 38. In some cases, the Tuohy seal 56 may be engaged (and closed), or disengaged (and opened) by rotating the proximal nut 60 in an appropriate rotational direction. In some cases, the Tuohy seal 56 may be considered as providing high-pressure hemostasis.

In some cases, the flat seal 44 may be engaged (and opened) by sliding the lever 40 in a direction indicated by an arrow 64, compressing the spring 42. In some cases, the lever 40 includes a pair of pins 66 that extend through slots (not visible in these Figures) formed within the hub body 38, allowing the pair of pins 66 to extend through slots 68 (one is visible) that are formed within the seal cavity 46 and engage pads 70 that are formed on an exterior of the plunger 50. As a result, sliding the lever 40 in the direction indicated by the arrow 64, against a biasing force applied by the spring 42, causes the plunger 50 to also move in the direction indicated by the arrow 64. The plunger 50 engages and opens the flat seal 44. When the lever 40 is released, the biasing force applied by the spring 42 pushes the lever 40 back to its original (relaxed) position. As the lever 40 moves in a direction opposite that indicated by the arrow 64, the plunger 50 also moves in a direction opposite that indicated by the arrow 64. This means that the plunger 50 withdraws from engagement with the flat seal 44, and the flat seal 44 is allowed to close. In some cases, the plunger 50 may be considered as being an example of an internal actuation mechanism that is engaged with an external actuation mechanism (the lever 40). In some instances, the spring 42 may be excluded, particularly if the flat seal 44 is made of an elastomeric material that itself can provide a biasing force against the plunger 50 being moved in the direction indicated by the arrow 64.

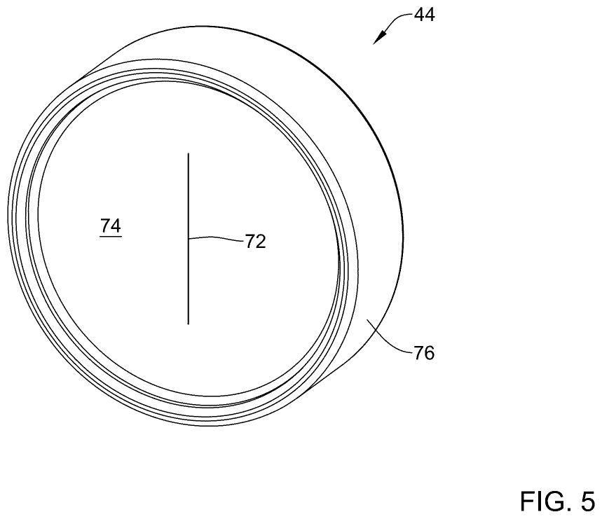

FIG. 5 is a perspective view of the flat seal 44. As can be seen, the flat seal 44 includes a slit 72 that extends across a face 74 of the flat seal 44. The flat seal 44 has a periphery 76 that extends around the flat seal 44 and that provides a circular profile that allows the flat seal 44 to fit within the seal cavity 46. The slit 72 is normally closed but can be opened by extending something through the slit 72. The flat seal 44 may be opened by extending the plunger 50 through the slit 72 by advancing the plunger 50 in the direction indicated by the arrow 64. The flat seal 44 may be formed of any suitable elastomeric material. In some cases, the flat seal 44 may be formed of an elastomeric polymer such as silicone.

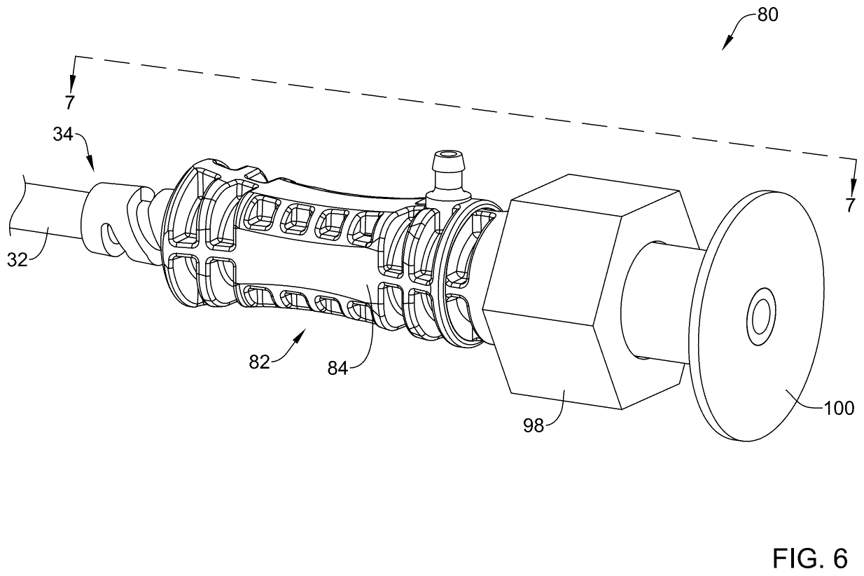

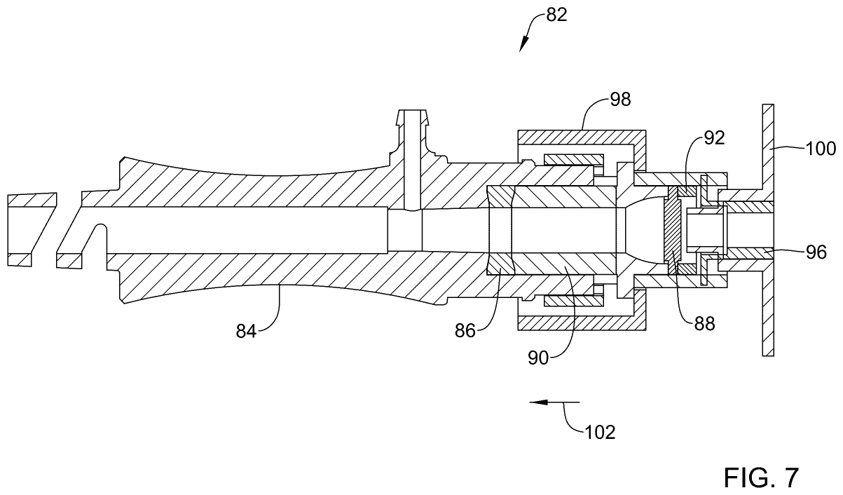

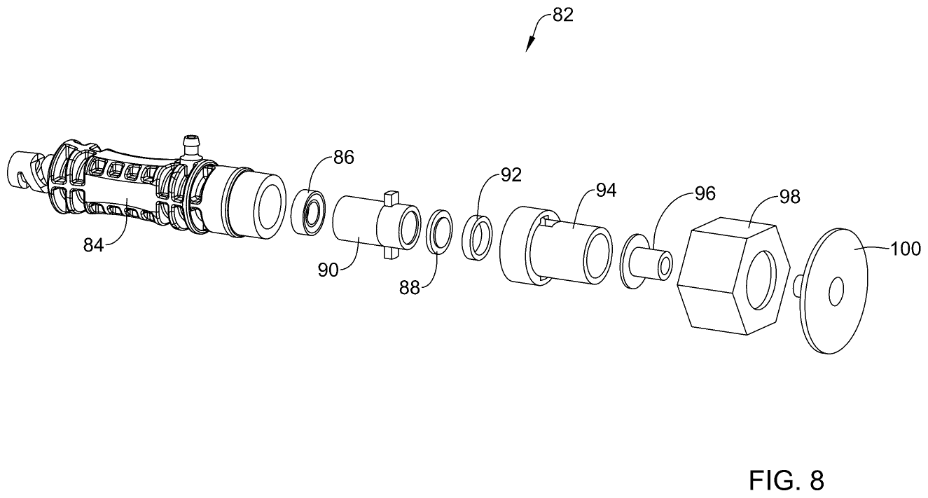

FIG. 6 is a perspective view of an illustrative guide catheter 80. The guide catheter 80 may be considered as being an example of the guide catheter 24 shown in FIG. 1. The guide catheter 80 includes the elongate shaft 32 that extends from the proximal region 34 to a distal region (as shown in FIG. 1). A guide catheter hub 82 is coupled to the proximal region 34 of the elongate shaft 32. The guide catheter hub 82 includes a hub body 84. FIG. 7 is a cross-sectional view of the guide catheter 80 taken along the line 7-7 of FIG. 6. FIG. 8 is an exploded perspective view of the guide catheter hub 82.

The guide catheter hub 82 includes a Tuohy seal 86 and a flat seal 88. A first cartridge 90 is disposed between the Tuohy seal 86 and the flat seal 88. A second cartridge 92 is disposed between the flat seal 88 and a cap 94. The guide catheter hub 82 includes a first pusher 96 that is positioned between the cap 94 and a nut 98. A second pusher 100 is positioned on an opposite side of the nut 98 from the first pusher 96. While not shown, in some cases, a spring may be located between the first pusher 96 and the second pusher 100 to bias the second pusher 100 to a position in which the second pusher 100 does not engage the flat seal 88. Pushing the second pusher 100 in a direction indicated by the arrow 102 will cause the second pusher 100 to engage with the flat seal 88. Releasing the second pusher 100 will allow the spring (not shown) to urge the second pusher 100 in a direction opposite that indicated by the arrow 102, thereby once again closing the flat seal 88.

In some cases, the Tuohy seal 86 may be closed by rotating the nut 98 to drive the first cartridge 90 towards the Tuohy seal 86. Rotating the nut 98 in an opposing direction will cause the first cartridge 90 to move away from the Tuohy seal 86, thus allowing the Tuohy seal 86 to open. While not shown, the nut 98 may have a threaded arrangement with a proximal end of the hub body 84, thereby allowing the nut 98 to be advanced in a direction indicated by an arrow 102 to engage the Tuohy seal 86 or to withdraw in a direction opposite that indicated by the arrow 102 to disengage the Tuohy seal 86. The flat seal 88 provides resistance to air ingress and permanent hemostasis while the Tuohy seal 86 provides high-pressure hemostasis.

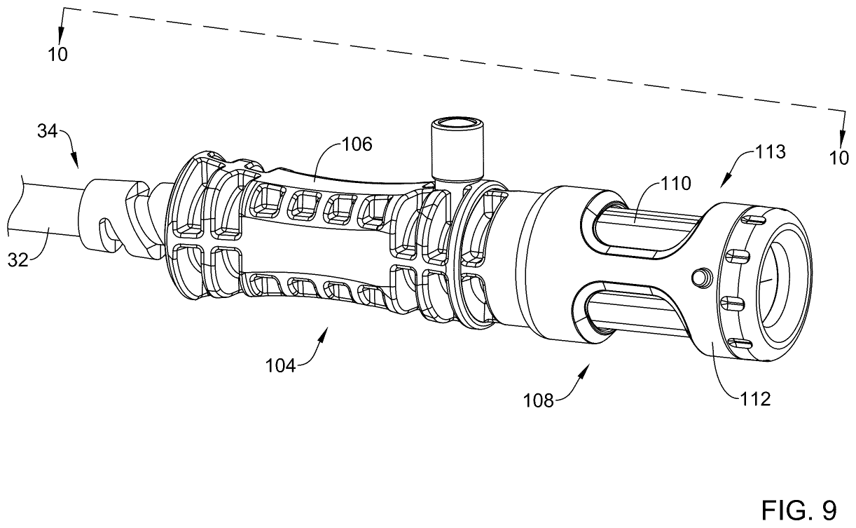

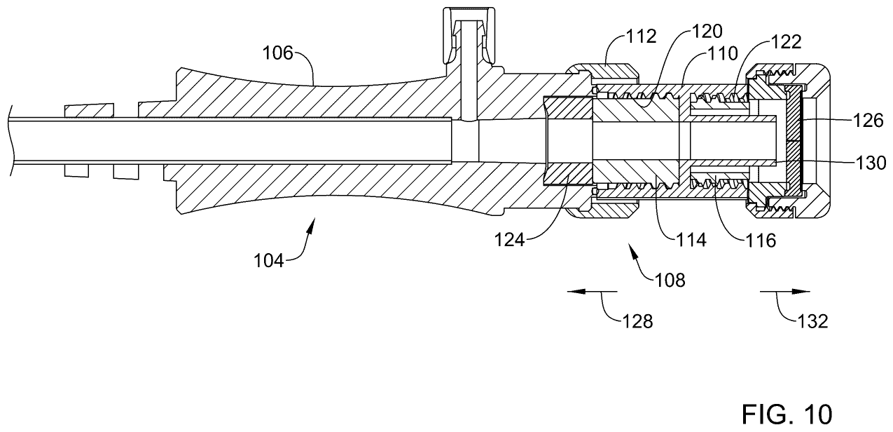

FIG. 9 is a perspective view of an illustrative guide catheter 104. The guide catheter 104 may be considered as being an example of the guide catheter 24 shown in FIG. 1. The guide catheter 104 includes the elongate shaft 32 that extends from the proximal region 34 to a distal region (as shown in FIG. 1). A guide catheter hub 104 is coupled to the proximal region 34 of the elongate shaft 32. The guide catheter hub 104 includes a hub body 106. An actuation hub 108 is disposed proximal of the hub body 106. FIG. 10 is a cross-sectional view of the guide catheter 104 taken along the line 10-10 of FIG. 9.

The actuation hub 106 includes a nut 110 that is disposed within a hub body 112. The hub body 112 includes voids 113 that allows a user to reach the nut 110 within the hub body 112. Inside the hub body 112 is a threaded distal pusher 114 and a threaded proximal pusher 116. The nut 110 has a first threaded surface 120 that is adapted to engage the threaded distal pusher 114 and a second threaded surface 122 that is adapted to engage the threaded proximal pusher 116. In some cases, a Tuohy seal 124 and a flat seal 126 are both disposed within the actuation hub 108. In some cases, rotating the nut 110 in a first rotational direction (clockwise, for example) causes the threaded distal pusher 114 to move in a direction indicated by an arrow 128 and engage and close the Tuohy seal 124. In some cases, rotating the nut 110 in a second rotational direction (counter-clockwise, for example) causes a proximally facing extension 130 to move in a direction indicated by an arrow 132, and thus engage and open the flat seal 126.

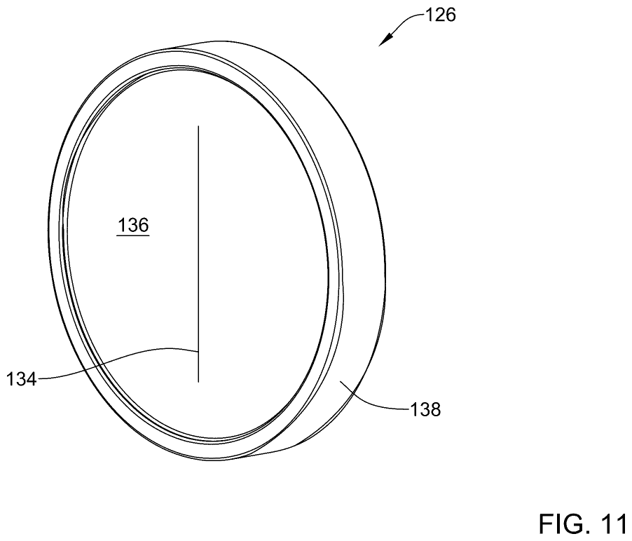

FIG. 11 is a perspective view of the flat seal 126. As can be seen, the flat seal 126 includes a slit 134 that extends across a face 136 of the flat seal 126. The flat seal 126 has a periphery 138 that extends around the flat seal 126 and that provides a circular profile that allows the flat seal 126 to fit within the actuation hub 108. The slit 134 is normally closed but can be opened by extending something through the slit 134. The flat seal 126 may be opened by extending the proximally facing extension 130 through the slit 134 by advancing the proximally facing extension 130 towards the flat seal 126. The flat seal 126 may be formed of any suitable elastomeric material. In some cases, the flat seal 126 may be formed of an elastomeric polymer such as silicone.

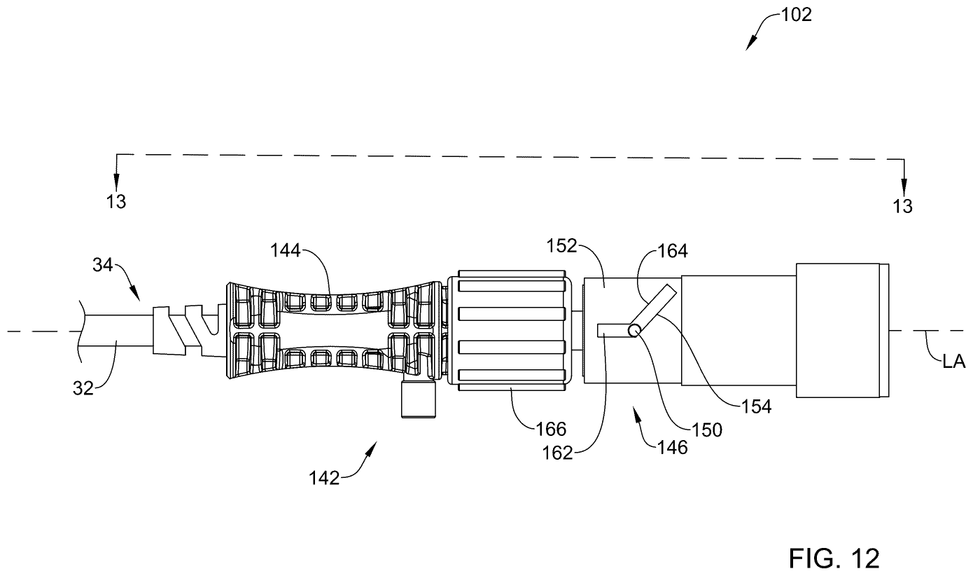

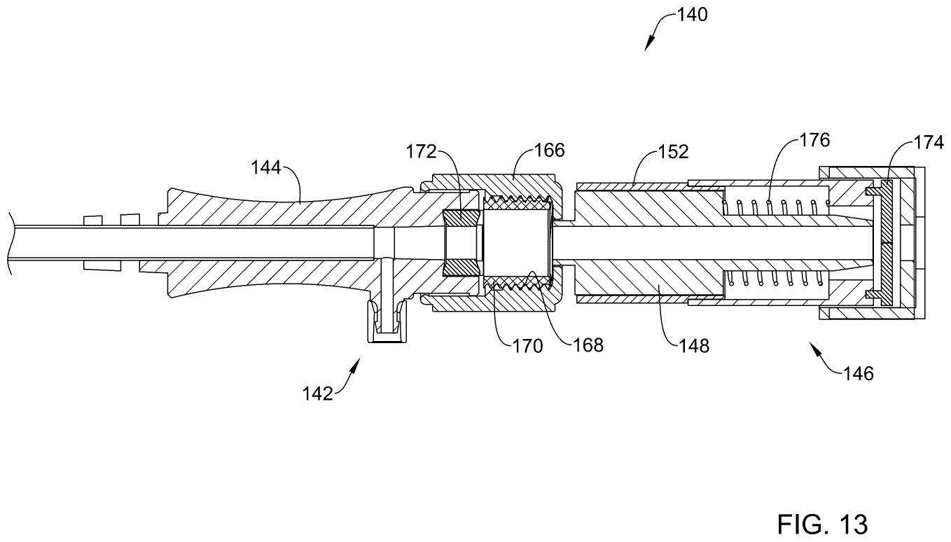

FIG. 12 is a perspective view of an illustrative guide catheter 140. The guide catheter 140 may be considered as being an example of the guide catheter 24 shown in FIG. 1. The guide catheter 140 includes the elongate shaft 32 that extends from the proximal region 34 to a distal region (as shown in FIG. 1). A guide catheter hub 142 is coupled to the proximal region 34 of the elongate shaft 32. The guide catheter hub 142 includes a hub body 144. An actuation hub 146 is disposed proximal of the hub body 144. FIG. 13 is a cross-sectional view of the guide catheter 140 taken along the line 13-13 of FIG. 12.

The actuation hub 146 includes an inner hub 148 having a tab 150 that extends radially outwardly from the inner hub 148. The inner hub 148 is disposed within an outer hub 152. The outer hub 152 includes a slot 154 that is adapted to allow the tab 150 to slide within the slot 154. In some cases, the slot 154 may be linear. In some cases, as shown, the slot 154 may have a boomerang shape, with a first portion 162 that is aligned with a longitudinal axis LA of the guide catheter 140 and a second portion 164 that extends at an acute angle relative to the longitudinal axis LA. In some cases, this permits a longer total effective length for the slot 154 relative to the geometry of the outer hub 152, for example.

The guide catheter 140 includes a nut 166 having a threaded surface 168 that is adapted to threadedly engage a threaded member 170 that is associated with the inner hub 148. In some cases, rotating the nut 166 in a first direction (such as clockwise) causes the threaded member 170 to translate and engage a Tuohy seal 172. Rotating the nut 166 in an opposing direction (such as counter-clockwise) causes the inner hub 148 to translate relative to the outer hub 152 as the tab 150 slides within the slot 154 and to engage and open a flat seal 174. In some cases, as shown, the Tuohy seal 172 may be disposed within the hub body 144 and the flat seal 174 may be disposed within the actuation hub 146.

In some cases, a spring 176 biases the inner hub 148 to a position (as shown) in which the inner hub 148 does not contact or otherwise engage the flat seal 174. When the nut 166 is released, the spring 176 pushes the inner hub 148 away from the flat seal 174, and the flat seal 174 is able to close. In some cases, depending on the resiliency of the flat seal 174, the spring 176 may not be necessary.

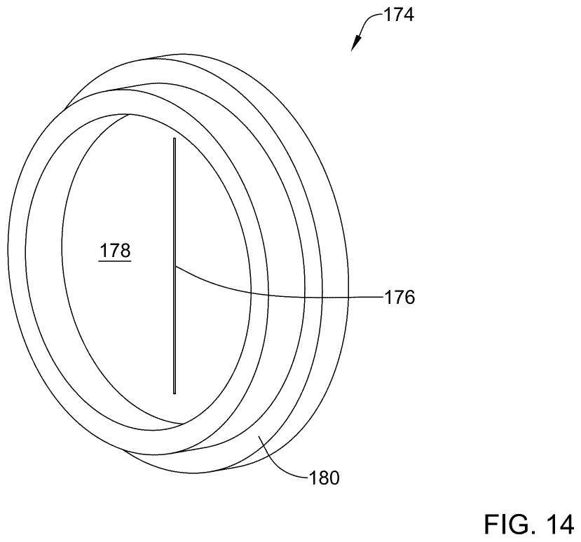

FIG. 14 is a perspective view of the flat seal 174. As can be seen, the flat seal 174 includes a slit 176 that extends across a face 178 of the flat seal 174. The flat seal 174 has a periphery 180 that extends around the flat seal 174 and that provides a circular profile that allows the flat seal 174 to fit within the actuation hub 146. The slit 176 is normally closed but can be opened by extending something through the slit 176. The flat seal 174 may be opened by extending the inner hub 148 through the slit 176 by advancing the inner hub 148 towards the flat seal 174. The flat seal 174 may be formed of any suitable elastomeric material. In some cases, the flat seal 174 may be formed of an elastomeric polymer such as silicone.

In comparing the flat seal 44, the flat seal 88, the flat seal 126 and the flat seal 174, it can be seen that there are variations among these flat seals. Some of these variations may be related to how the particular flat seals 44, 88, 126, 174 are packed within their respective guide catheters 30, 80, 102 and 140. Similarities between the flat seals 44, 88, 126, 174 include that each of the flat seals 44, 88, 126, 174 are formed of an elastomeric material such as but not limited to silicone, and that each of the flat seals 44, 88, 126, 174 include a normally closed slit 72, 134 and 176. As a result, each of the flat seals 44, 88, 126, 174 are able to prevent air ingress.

The materials that can be used for the devices described herein may include those commonly associated with medical devices. The devices described herein, or components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel and various nickel alloys; combinations thereof; and the like; or any other suitable material.

A sheath or covering (not shown) may be disposed over portions or all of the devices described herein in order to define a generally smooth outer surface. In other embodiments, however, such a sheath or covering may be absent. The sheath may be made from a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the devices described herein may be sandblasted, beadblasted, sodium bicarbonate-blasted, electropolished, etc. In these as well as in some other embodiments, a coating, for example a lubricious, a hydrophilic, a protective, or other type of coating may be applied. Alternatively, a sheath may include a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

Portions of the devices described herein may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. Those skilled in the art will recognize that these materials can vary widely without deviating from the scope of the present disclosure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A guide catheter for accommodating a left atrial appendage closure (LAAC) device being advanced towards a left atrial appendage (LAA), the guide catheter comprising:
an elongate shaft extending from a proximal region to a distal region;
a guide catheter hub coupled to the proximal region of the elongate shaft, the guide catheter hub including:
a hub body;
a Tuohy seal disposed relative to the hub body;
a flat seal disposed relative to the hub body; and
a mechanism disposed relative to the hub body, the mechanism adapted to releasably open the flat seal.

2. The guide catheter of claim 1, wherein the flat seal is disposed within the hub body.

3. The guide catheter of claim 1, wherein the mechanism is disposed within the hub body.

4. The guide catheter of claim 3, wherein the mechanism comprises:
an internal actuation mechanism disposed within the hub body; and
an external actuation mechanism disposed outside of the hub body, the external actuation mechanism adapted to engage with and move the internal actuation mechanism.

5. The guide catheter of claim 4, wherein the internal actuation mechanism is biased to a position in which the flat seal is closed.

6. The guide catheter of claim 5, further comprising a biasing member that is adapted to bias the internal actuation mechanism to the position in which the flat seal is closed.

7. The guide catheter of claim 1, further comprising a proximal nut adapted to reversibly close the Tuohy seal.

8. The guide catheter of claim 1, further comprising an actuation hub disposed proximal of the hub body.

9. The guide catheter of claim 8, wherein the Tuohy seal and the flat seal are both disposed within the actuation hub.

10. The guide catheter of claim 9, wherein the mechanism comprises:
a threaded distal pusher;
a threaded proximal pusher; and
a nut having a first threaded surface adapted to engage the threaded distal pusher and a second threaded surface adapted to engage the threaded proximal push, the nut including a proximally facing extension;
wherein moving the nut in a first direction causes the threaded distal pusher to engage and close the Tuohy seal; and
wherein moving the nut in a second direction causes the proximally facing extension to engage and open the flat seal.

11. The guide catheter of claim 8, wherein the Touhy seal is disposed within the hub body and the flat seal is disposed within the actuation hub.

12. The guide catheter of claim 11, wherein the actuation hub comprises:
an inner hub including a tab extending radially outwardly from the inner hub;
an outer hub accommodating the inner hub within the outer hub, the outer hub including a slot adapted to allow the tab to slide within the slot.

13. The guide catheter of claim 12, wherein the mechanism comprises:
a nut having a threaded surface;
a threaded member associated with the inner hub;
wherein rotating the nut in a first direction causes the threaded member to engage and close the Tuohy seal; and
wherein rotating the nut in an opposing section direction causes the inner hub to translate relative to the outer hub as the tab slides within the slot, the inner hub engaging and opening the flat seal.

14. The guide catheter of claim 13, wherein the mechanism further comprises a spring that biases the inner hub to a position in which the inner hub does not engage the flat seal.

15. A guide catheter for accommodating a left atrial appendage closure (LAAC) device being advanced towards a left atrial appendage (LAA), the guide catheter comprising:
an elongate shaft extending from a proximal region to a distal region;
a guide catheter hub coupled to the proximal region of the elongate shaft, the guide catheter hub including:
a hub body;
a first seal disposed within the hub body, the first seal adapted to avoid air ingress;
an internal actuation mechanism disposed within the hub body, the internal actuation mechanism adapted to reversibly open the first seal;
a second seal disposed within the hub body, the second seal adapted to provide high pressure hemostasis; and
an external actuation mechanism that is adapted to engage with and move the internal actuation mechanism.

16. The guide catheter of claim 15, wherein the internal actuation mechanism is biased to a position in which the first seal is closed.

17. The guide catheter of claim 15, wherein the internal actuation mechanism is further adapted to reversibly close the second seal.

18. The guide catheter of claim 15, further comprising a proximal nut adapted to reversibly close the second seal.

19. A guide catheter for accommodating a left atrial appendage closure (LAAC) device being advanced towards a left atrial appendage (LAA), the guide catheter comprising:
an elongate shaft extending from a proximal region to a distal region;
a guide catheter hub coupled to the proximal region of the elongate shaft;
an actuation hub disposed proximally of the guide catheter hub;
a Tuohy seal disposed relative to the hub body;
a flat seal disposed within the actuation hub; and
a mechanism adapted to selectively engage the Tuohy seal or the flat seal.

20. The guide catheter of claim 19, wherein:
actuating the mechanism in a first direction causes the mechanism to engage and close the Tuohy seal; and
actuating the mechanism in a second direction causes the mechanism to engage and open the flat seal.
